(19) United States
(12) Patent Application Publication
(10) Pub. No.: US 2011/0046223 A1
(43) Pub. Date: Feb. 24, 2011

(54) TREATMENT OF NEUROFIBROMATOSIS

(75) Inventors: Yoel Kloog, Herzliya (IL); Shiran Kringel, Petah-Tikva (IL); Eitan Friedman, Tel-Aviv (IL); Reuven Stein, Ramat HaSharon (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: RAMOT AT TEL-AVIV UNIVERSITY LTD., Tel-Aviv (IL)

(21) Appl. No.: 12/308,450

(22) PCT Filed: Jun. 14, 2007
(86) PCT No.: PCT/IL2007/000725

§ 371 Date: Oct. 29, 2010

Related U.S. Application Data
(60) Provisional application No. 60/813,642, filed on Jun. 14, 2006.

(51) Int. Cl. A61K 31/192; A61P 17/00; A61P 25/00; A61P 19/00; A61P 21/00

(52) U.S. Cl. 514/568

(57) ABSTRACT

Disclosed are methods of treating neurofibromatosis by administering to a human in need thereof effective amounts of FTS, or various analogs thereof, or a pharmaceutically acceptable salt thereof, optionally, in combination with colchicine. Also disclosed are pharmaceutical compositions comprising FTS, or various analogs thereof, or a pharmaceutically acceptable salt thereof; colchicine; and a pharmaceutically acceptable carrier.

### CROSS-REFERENCE TO RELATED APPLICATIONS

This application claims the benefit of the filing date of U.S. Provisional Patent Application No. 60/813,642 filed Jun. 14, 2006, the disclosure of which is hereby incorporated herein by reference.

### GOVERNMENT RIGHTS

The work leading to the invention described herein was funded in part by the Department of the Army under the U.S. Army Medical Research Acquisition Activity (USAMRAA): Contract No. W81XWH-04-1-0207. Therefore, the government may have certain rights in the invention.

### BACKGROUND OF THE INVENTION

Neurofibromatosis type I (NF-1), also known as von Recklinghausen's neurofibromatosis or peripheral neurofibromatosis, is an inherited condition, which involves development of changes in the nervous system, skin, bones, and muscles manifested by the presence of multiple soft nodules, neurofibromas, and is associated with hyper-pigmented (café-au-lait) spots. The hallmark lesion of NF-1 is the plexiform neurofibroma. These lesions are composed of sheets of neurofibromatous tissue which may infiltrate and encase major nerves, blood vessels, and other vital structures. [See Rasmussen, S. A., et al., Am J Hum Genet. 68(5): 1110-1118 (2001)].

NF-1 is an unpredictable disorder and its severity tends to vary widely. Patients with NF-1 are predisposed, to develop a variety of tumors including Schwannomas or malignant peripheral nerve sheath tumors (MPNSTs), astrocytic brain tumors (glioblastomas), and pheochromocytomas. [Cichowski, K., Jacks, T., Cell 104:593-604 (2001); Korf, B. R., Am J Med Genet. 85:31-7 (1999)]. Those affected by the more severe variants of NF-1 may have a shorter life expectancy if the disease is associated with CNS tumors or other malignancies, mental retardation, or severe seizures.

The disorder is caused by a mutation on the long arm of chromosome 17 which encodes a protein known as neurofibromin, which is a tumor suppressor protein and plays an important role in intracellular signaling. More specifically, neurofibromin facilitates the hydrolysis and inactivation of active Ras-GTP. [Ballester, R. et al., Cell 63:851-9 (1990); Martin, G. A. et al., Cell 63:843-9 (1990); Xu, G. F., et al., Cell 62:599-608 (1990)]. Deficiency of neurofibromin results in an increase of activated Ras-GTP, which, through stimulation of the Ras signal transduction cascade, contributes to the etiology of NF-1. [Basu, T. N., et al., Nature 356:713-5 (1992); DeClue, J. E., et al., Cell 69: 265-73 (1992)].

The mutant gene coding for the protein neurofibromin is transmitted with an autosomal dominant pattern of inheritance. However, up to 50% of NF-1 cases arise due to spontaneous mutation. The disease affects about 1 in 3,500 individuals. [Stumpf, D. A., et al., Arch. Neurol. 45:575-578 (1988)].

The diagnostic criteria for NF-1 according to a National Institutes of Health (NIH) Consensus Development Conference include the presence of two or more of the following: (1) six or more café-au-lait macules more than 5 mm in prepubertal individuals or 15 mm in greatest diameter in postpubertal individuals; (2) two or more neurofibromas of any type, or one plexiform neurofibroma; (3) freckling in the axillary or inguinal regions; (4) optic glioma; (5) two or more Lisch nodules (iris hamartomas); (6) a distinctive bony lesion such as sphenoid dysplasia or thinning of long-bone cortex, with or without pseudoarthrosis; (7) a first-degree relative with NF-1. [Stumpf, D. A., et al., supra.].

Therapy for a patient with NF-1 is aimed at palliating symptoms and improving quality of life. Treatment modalities typically include radiation therapy, chemotherapy, and surgical resection or decompression of an enlarging lesion. [Smirniotopoulos, J., Radiologic Pathology, 2nd ed (2003-2004); Cotran, R., Kumar, V., Robbins, S. (eds). Robbins Pathologic Basis of Disease, 5th ed. (1994)].

### SUMMARY OF THE INVENTION

A first aspect of the present invention is directed to a method of treating neurofibromatosis. The method comprises administering to a human in need thereof (e.g., a human diagnosed with or having neurofibromatosis) an effective amount of S-farnesylthiosalicylic acid (FTS) or an analog thereof, or a pharmaceutically acceptable salt thereof.

Another aspect of the present invention is directed to a method of treating a neurofibromatosis. The method comprises administering to a human in need thereof effective amounts of S-farnesylthiosalicylic acid (FTS) or an analog thereof, or a pharmaceutically acceptable salt thereof, and colchicine.

A further aspect of the present invention is directed to a pharmaceutical composition useful in the treatment of neurofibromatosis. The composition comprises effective amounts of FTS or an analog thereof or a pharmaceutically acceptable salt thereof; colchicine; and a carrier. Methods of making the compositions are further provided.

The results of a first set of experiments described herein showed that the Ras inhibitor, FTS, reversed the cellular manifestations associated with neurofibromatosis (NF-1) in a neurofibromin-deficient human cell line and inhibited NF-1 associated tumor growth in a rodent model.

The results of a further set of experiments described herein showed that combining colchicine, in the presence of FTS sensitized colchicine-treated cells and thus, enhanced colchicine-induced cell death in neurofibromin-deficient NF-1 rodent cells. Similarly, in a neurofibromin-deficient human cell line, combination treatment with FTS and colchicine, enhanced colchicine-induced cell death. In contrast, colchicine-induced cell death was not enhanced by FTS in neither rodent nor human cells that normally expressed neurofibromin (i.e., non-NF-1 cells).

### DESCRIPTION OF THE DRAWINGS

FIG. 1A is a sequence analysis of the region of exon 7 in the 90-8 cell line, in which the C910T nonsense mutation of codon 304 (R304X) was detected (labeled by a square).

FIG. 1B are the allelic patterns of the 90-8 cell line using D17S250 marker (left) showing retention of heterozygosity, and D17S1166 (right) consistent with loss of heterozygosity. Allele sizes (in base-pairs) are shown above the relevant peaks.

FIG. 1C are immunoblots illustrating amounts of neurofibromin p120 RasGAP in NF-1 (ST88-14, 90-8, T265P21) and non-NF-1 MPNST (STS26T) cells (left). Total rat brain homogenate (serving as standard) is depicted (right).

FIG. 1D are immunoblots illustrating the amounts of Ras-GTP expressed as percentages of total Ras in the various cell lines (means±SD, n=6).

FIG. 2A are photomicrograph images of vehicle treated (control) and FTS-treated NF-1 (ST88-14, 90-8, T265P21) and non-NF-1 (STS26T) cells.

FIG. 2B is a graph illustrating dose-response curves of inhibition for NF-1 and non-NF-1 cells at increasing concentration of FTS (μM), expressed as a percentage of the cell numbers in control cultures (means±SD, n=4).

FIG. 2C is a graph illustrating the correlations between IC₅₀ values (means±SD, n=4), evaluated from the FTS dose-dependent inhibition curves shown in FIG. 2B, and the normalized Ras-GTP levels (arbitrary units) evaluated from experiments, as shown in FIG. 1D (means±SD, n=9). R² is the correlation coefficient.

FIG. 3A is an immunoblot illustrating the effects of FTS on Ras and Ras-GTP levels (left). The bar graph (right) depicts total Ras and Ras-GTP levels in the FTS-treated cultures (means±SD, n=6).

FIG. 3B is an immunoblot illustrating the effects of FTS on the levels of phospho-ERK, phospho-Akt, and RalA-GTP (left). The bar graph (right) depicts total Ral-GTP, P-ERK, and P-Akt levels in the FTS-treated cultures (means±SD, n=6).

FIGS. 4A-4D are immunoblots illustrating the relative amounts of Ras, Ras-GTP, ERK, phosphor-ERK, Akt, phosphor-Akt, and tubulin (loading control) in each of the non-NF-1 [STS26T (A)] and NF-1 cell lines [T265P21 (B)], [ST88-14 (C)], [90-8 (D)].

FIG. 5A are images taken by confocal fluorescence microscopy illustrating the relative amounts of actin stress fibers in vehicle-treated (control) and FTS-treated NF-1 (ST88-14, 90-8, T265P21) and non-NF-1 (STS26T) cells.

FIG. 5B is a bar graph illustrating the statistical analysis of cells containing stress fibers expressed as a percentage of the total cell number (means±SD, n=3, *P<0.01, **P<0.05).

FIG. 6A are photomicrograph images (left) of anchorage-independent cell growth in soft agar in vehicle-treated (control) and FTS-treated NF-1 cells (ST88-14) and non-NF-1 cells (STS26T). The bar graphs (right) depict the statistical analysis of each, respectively (means±SD, n=9, *P<0.001).

FIG. 6B are two bar graphs illustrating inhibition of ST88-14-induced tumor growth at varying concentrations of FTS, either administered i.p. (left) or oral (right). The bar graphs further illustrate tumor weight [means±SD, n=6, in the i.p.-dosing, and n=10 in the oral-dosing experiments (*P<0.05; **P<0.01; ***P<0.001)].

FIG. 7 is a bar graph illustrating the effects of FTS on colchicine-induced death for NF-1^{-/-}, NF-1^{+/-}, and NF-1^{+/+} MEF cells and human ST-88 and STS26T cells.

### DETAILED DESCRIPTION

Ras proteins act as on-off switches that regulate signal-transduction pathways controlling cell growth, differentiation, and survival. [Reuther, G. W., Der, C. J., Curr Opin Cell Bioi. 12:157-65 (2000)]. They are anchored to the inner leaflet of the plasma membrane, where activation of cell-surface receptors, such as receptor tyrosine kinase, induces the exchange of guanosine diphosphate (GDP) for guanosine triphosphate (GTP) on Ras and the conversion of inactive Ras-GDP to active Ras-GTP. [Scheffzek, K., Ahmadian, M. R., Kabsch, W. et al. Science 277:333-7 (1997)]. The active Ras protein promotes oncogenesis through activation of multiple Ras effectors that contribute to deregulated cell growth, differentiation, and increased survival, migration and invasion. [See e.g., Downward, J., Nat. Rev. Cancer 3:11-22 (2003); Shields, J. M., et al., Trends Cell Biol 20:147-541 (2000); and Mitin, N., et al., Curr Biol 15:R563-74 (2005)].

FTS is a potent Ras inhibitor that acts in a rather specific manner on the active, GTP-bound forms of H-, N-, and K-Ras proteins. [Weisz, B., Giehl, K., Gana-Weisz, M., Egozi, Y., Ben-Baruch, G., Marciano, D., Gierschik, P., Kloog, Y., Oncogene 18:2579-2588 (1999); Gana-Weisz, M., Halaschxek-Wiener, J., Jansen, B., Elad, G., Haklai, R., Kloog, Y., Clin. Cancer Res. 8:555-65 (2002)]. FTS competes with Ras-GTP for binding to specific saturable binding sites in the plasma membrane, resulting in mislocalization of active Ras and facilitating Ras degradation. [Haklai, et al., Biochemistry 37(5): 1306-14 (1998)]. This competitive inhibition prevents active Ras from interacting with, its prominent downstream effectors and results in reversal of the transformed phenotype in transformed cells that harbor activated Ras. As a consequence, Ras-dependent cell growth and transforming activities, both in vitro and in vivo, are strongly inhibited by FTS. [Weisz, B., et al., supra.; Gana-Weisz, M., et al., supra.].

FTS and its analogs useful in the present invention are represented by formula I:

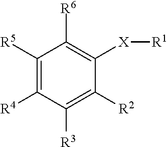

wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S.

The structure of FTS is as follows:

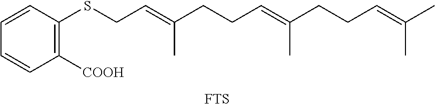

FTS analogs embraced by formula I include 5-fluoro-FTS, 5-chloro-FTS, 4-chloro-FTS, S-farnesyl-thiosalicylic acid methyl ester (FTSME), and S-geranyl, geranyl-thiosalicylic acid (GGTS). Structures of these compounds are set forth below.

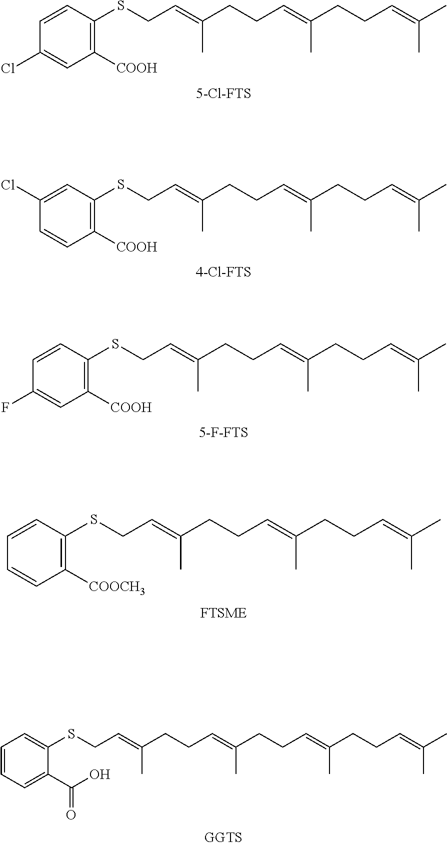

Methods for preparing the compounds of formula I are disclosed in U.S. Pat. Nos. 5,705,528 and 6,462,086. See also, Marom, M., Haklai, R., Ben-Baruch, G., Marciano, D., Egozi, Y., Kloog, Y. J Biol Chem 270:22263-70 (1995).

Pharmaceutically acceptable salts of the Ras antagonists of formula I may be useful. These salts include, for example, sodium and potassium salts. Other pharmaceutically acceptable salts may be selected in accordance with standard techniques as described in Berge, S. M., Bighley, L. D., and Monkhouse, D. C., J. of Pharm. Sci. 66(1):1-19 (1977). In preferred embodiments, however, FTS and its analogs are not administered in the form of a salt (i.e., they are administered in non-salified form).

In some embodiments, treatment includes administering colchicine. Colchicine [Acetamide, N-((7S)-5,6,7,9-tetrahydro-1,2,3,10-tetramethoxy-9-oxobenzo(a)heptalen-7-yl)-] has the chemical formula C₂₂H₂₅NO₆ and inhibits microtubule polymerization by binding to tubulin, one of the main constituents of microtubules. Tubulin (MW approximately 10,000 Dalton) is a protein consisting of two forms, alpha and beta. Alpha- and beta-tubulin form dimmers that polymerize to form long filaments of microtubules. When colchicine binds to the tubulin dimers, the dimers are unable to form the microtubules. The microtubules are vital for formation of spindle fibers during mitosis and meiosis, intracellular transport of vesicles and proteins, flagella reassembly, ameboid motility, and other cellular processes. Apart from inhibiting mitosis, a process heavily dependent on cytoskeletal changes, it also inhibits neutrophil motility and activity, leading to a net anti-inflammatory effect.

Methods of preparing and using colchicine are well-known in the art. See e.g., Remington's Pharmaceutical Sciences and U.S. Pharmacopeia: National Formulary, Mack Publishing Company, Easton, Pa. (1990). Colchicine is FDA-approved for the treatment of gout and also for familial Mediterranean fever, secondary amyloidosis, and scleroderma.

Formulations for the active(s) and administration thereof are accomplished in accordance with the following teachings. The frequency of administration, dosage amounts, and the duration of treatment of each of the active agents may be determined depending on several factors, e.g., the overall, health, size and weight of the patient, the severity of the disease, the patient's tolerance to the treatment, and the particular treatment regimen being administered. For example, duration of treatment with FTS or the combination of FTS and colchicine may last a day, a week, a year, or until remission of the disease is achieved. Thus, relative timing of administration of these active agents is not critical (e.g., FTS may be administered before, during, and after treatment with colchicine).

As used herein, the term “effective amount” refers to the dosage(s) of FTS alone or in combination with colchicine that are effective for the treating, and thus includes dosage amounts that ameliorate symptom(s) of the disorder and its associated manifestations, diminish extent of disease, delay or slow disease progression, or achieve partial or complete remission or prolong survival. The average daily dose of FTS generally ranges from about 50 mg to about 2000 mg, and in some embodiments, ranges from about 200 mg to about 1600 mg. According to Phase I human clinical trials (for various cancers) conducted by Concordia Pharmaceuticals, Inc., S-Farnesylthiosalicylic acid (FTS, Salirasib) is a relatively safe compound with no dose-limiting toxicities at doses up to 1800 mg/day. The average daily dose of colchicine generally ranges from about 0.25 mg to about 2.50 mg, and in some embodiments, ranges from about 0.25 mg to about 1.5 mg.

In some embodiments, both drugs are administered on a daily basis, e.g., each in single once-a-day or divided doses or each in the same dosage form. They may be administered at the same or different times. In other embodiments, the doses may be administered in different dosage forms, e.g., as a tablet, a capsule, or an injection. In other embodiments, each drug is administered two or more times per day.

FTS may be administered in accordance with standard methods. In preferred embodiments, FTS is administered orally. In some embodiments, FTS may be administered by dosing orally on a daily basis for three weeks, followed by a one-week “off period”, and repeating until remission is achieved. In another embodiment, FTS may be administered by dosing twice daily and continuing the treatment until remission is achieved. Parenteral administration is also suitable.

In some embodiments, colchicine is administered orally. In some embodiments, colchicine may be administered by dosing orally, on a daily basis, one or more times per day, or until remission is achieved. Parenteral administration is also suitable.

In some embodiments, the administration of FTS with colchicine may be cyclic. For example, in one treatment regimen, FTS (200 mg) is administered twice daily for a period of three weeks followed by a one-week interval without FTS (“off period”) while colchicine (0.50 mg) is administered once daily for a week and then one tablet twice daily and continuously (e.g., without an “off period”). The treatment regimen is repeated as many times as needed, e.g., until remission is achieved. Under this regimen, colchicine is administered continuously (with increasing dose amounts as needed) while the FTS is administered in three-week cycles each separated by a one-week “off period”.

The treatment regimen may entail administration with FTS and colchicine continuously without interruption (i.e., without an “off period”) until remission is achieved. Some embodiments may involve administering to a patient in need thereof both actives in the same dosage form, (e.g., a capsule or a tablet) twice, or thrice daily depending on the prescribed treatment schedule. For example, one schedule prescribes FTS (200 mg)/colchicine (0.25 mg) or FTS (200 mg)/colchicine (0.50 mg) twice daily in tablet form. Another schedule prescribes FTS (100 mg)/colchicine (0.25 mg) thrice daily in capsule form. Yet another schedule prescribes FTS (300 mg)/colchicine (0.25 mg) twice daily in capsule form. Alternatively, the actives are administered in separate dosage forms, (e.g., one as a capsule and the other a tablet) daily and substantially simultaneously.

Compositions for use in the present invention (which can contain either or both active pharmaceutical agents) can be prepared by bringing the agent(s) into association with (e.g., mixing with) a pharmaceutically acceptable carrier. Suitable carriers are selected based in part on the mode of administration. Carriers are generally solid or liquid. In some cases, compositions may contain solid and liquid carriers. Compositions suitable for oral administration that contain either or both actives are preferably in solid dosage forms such as tablets (e.g., including film-coated, sugar-coated, controlled or sustained release), capsules, e.g., hard gelatin capsules (including controlled or sustained release) and soft gelatin capsules, powders and granules. The oral compositions, however, may be formulated in other carriers that, enable administration to a patient in other oral forms, e.g., a liquid or gel. Regardless of the form, the composition is divided into individual or combined doses containing predetermined quantities of the active ingredient(s).

Oral dosage forms may be prepared by mixing the active pharmaceutical ingredient or ingredients with one or more appropriate carriers (optionally with one or more other pharmaceutically acceptable additives or excipients), and then formulating the composition into the desired dosage form e.g., compressing the composition into a tablet or filling the composition into a capsule or a pouch. Typical carriers and excipients include bulking agents or diluents, binders, buffers or pH adjusting agents, disintegrants (including crosslinked and super disintegrants such as croscarmellose), glidants, and/or lubricants, including lactose, starch, mannitol, microcrystalline cellulose, ethylcellulose, sodium carboxymethylcellulose, hydroxypropylmethylcellulose, dibasic calcium phosphate, acacia, gelatin, stearic acid, magnesium stearate, corn oil, vegetable oils, and polyethylene glycols. Coating agents such as sugar, shellac, and synthetic polymers may be employed, as well as colorants and preservatives. See, Remington's Pharmaceutical Sciences, The Science and Practice of Pharmacy, 20th Edition, (2000).

In an oral dosage form, the FTS is typically present in a range of about 50 mg to about 500 mg, and in some embodiments, from about 100 mg to about 300 mg. In an oral dosage form colchicine is typically present in a range of about 0.25 mg to about 2.0 mg, and in some embodiments, from about 0.25 mg to about 0.50 mg.

Liquid form compositions include, for example, solutions, suspensions, emulsions, syrups, elixirs and pressurized compositions. The active ingredient(s), for example, can be dissolved or suspended in a pharmaceutically acceptable liquid carrier such as water, an organic solvent (and mixtures thereof), and/or pharmaceutically acceptable oils or fats. Examples of liquid carriers for oral administration include water (particularly containing additives as above, e.g., cellulose derivatives, preferably in suspension in sodium carboxymethyl cellulose solution), alcohols (including monohydric alcohols (including monohyclric alcohols and polyhydric alcohols, e.g., glycerin and non-toxic glycols) and their derivatives, and oils (e.g., fractionated coconut oil and arachis oil). The liquid composition can contain other suitable pharmaceutical additives such as solubilizers, emulsifiers, buffers, preservatives, sweeteners, flavoring agents, suspending agents, thickening agents, colorants, viscosity regulators, stabilizers or osmoregulators.

Carriers suitable for preparation of compositions for parenteral administration include Sterile Water for Injection, Bacteriostatic Water for Injection, Sodium Chloride Injection (0.45%, 0.9%), Dextrose Injection (2.5%, 5%, 10%), Lactated Ringer's Injection, and the like. Dispersions can also be prepared in glycerol, liquid polyethylene glycols and mixtures thereof, and in oils. Compositions may also contain tonicity agents (e.g., sodium chloride and mannitol), antioxidants (e.g., sodium bisulfite, sodium metabisulfite and ascorbic acid) and preservatives (e.g., benzyl alcohol, methyl paraben, propyl paraben and combinations of methyl and propyl parabens).

In order to fully illustrate the present invention and advantages thereof, the following specific examples/experiments are given, it being understood that the same is intended only as illustrative and in no way limitative.

### Example 1

### Experimental Design

The purpose of these in vitro and in vivo experiments was to assess the ability of the Ras inhibitor, FTS, to reverse the transformed phenotype of human NF-1-associated tumor cell lines [malignant peripheral nerve sheath tumor (MPNST) cells]. Here, the effects of FTS on the Ras-signaling cascade and on biochemical and phenotypic characteristics in neurofibromin-deficient human NF-1 MPNST cell lines ST88-14, T265P21 and 90-8 and the non-NF-1 human MPNST cell line STS26T were examined. The primary goal was to determine: (I) whether the NF-1-associated MPNST cell lines exhibited the pathogenic NF-1 gene mutations and neurofibromin deficiency; (II) whether steady-state levels of Ras-GTP correlated positively with neurofibromin deficiency and growth inhibition by FTS; (III) whether FTS down-regulated Ras-GTP and inhibited signaling in NF-1 cell lines; (IV) whether FTS restored an attenuated Ras signal-termination in NF-1 cells; (V) whether FTS affected cytoskeleton reorganization in NF-1 cells; (VI) whether FTS inhibited the anchorage-independent growth of ST88-14 and STS26T NF-1 cells; and (VII) whether FTS inhibited tumor growth as elicited by ST88-14 cells in a nude mouse model.

The results of the first set of experiments (I) established that a well-known mutation in NF-1 patients was present in the 90-8 cell line and in ST88-14 cells (data not shown). Additional results established that ST88-14, 90-8, and T265P21 cells were deficient in neurofibromin and non-NF-1 STS26T cells expressed significant amounts of neurofibromin.

In the second set of experiments (II), the results indicated that the relative deficiency of neurofibromin in each of the NF-1-derived cells was consistent with the observed levels of active GTP-bound Ras (steady-state levels) of each. Both NF-1 and non-NF-1 cells responded to FTS in a dose-dependent manner and the inhibition correlated positively with the neurofibromin deficiency.

The third set of experiments (III) revealed that FTS reduced the steady-state levels of Ras-GTP and its active downstream targets in NF-1 cells. Thus, FTS down-regulated active Ras in NF-1 cells.

The results of the fourth set of experiments (IV) demonstrated that FTS restored an attenuated Ras-signal termination in NF-1 cells. In all of the NF-1 cell lines, FTS not only reduced the serum-stimulated increases in Ras-GTP, RalA-GTP, phospho-ERK, and phospho-Akt levels but also, shortened the duration of the signals.

In the fifth set of experiments (V), FTS altered the cytoskeleton reorganization in NF-1 cells. All of the NF-1 cells treated with FTS exhibited a decrease in the numbers of cells with stress fibers, thus, demonstrating a reversal of the transformed phenotype of NF-1 cells by the Ras inhibitor FTS.

The results of the sixth set of experiments (VI) revealed that FTS inhibited the anchorage-independent growth of ST88-14 and STS26T NF-1 cells.

Finally, in the seventh set of experiments (VII), FTS inhibited tumor growth in nude mice implanted with ST88-14 cells. Both i.p. and oral dosing of FTS inhibited tumor growth.

### Materials and Methods

FTS was provided by Concordia Pharmaceuticals, Inc. (Ft. Lauderdale, Fla.). Colchicine was obtained from Biological Industries (Kibbutz Beit Haemek 2115, Israel (cat #12-003-1C). The enhanced chemiluminescence (ECL) kit was purchased from Amersham (Arlington Heights, Ill.); mouse anti-pan-Ras Ab (Ab-3) was from Calbiochem (La Jolla, Calif.); mouse anti-phospho-ERK Ab and mouse anti-tubulin Ab (AK-15) were from Sigma-Aldrich (St. Louis, Mo.); rabbit anti-ERK Ab and rabbit anti-neurofibromin Ab were from Santa Cruz Biotechnology (Santa Cruz, Calif.); rabbit anti-Akt Ab and rabbit anti-phospho-Akt (ser473; 4E2) Ab were from Cell Signaling Technology (Beverly, Mass.); mouse anti-p120 RasGAP Ab was from Upstate Biotechnology (Lake Placid, N.Y.), and mouse anti-RalA Ab was from Transduction Laboratories (Lexington, Ky.). Peroxidase goat anti-mouse IgG and peroxidase goat anti-rabbit IgG were from Jackson ImmunoResearch Laboratories (West Grove, Pa.).

### Genetic Analysis of the NF-1 Gene

Using high-molecular-weight DNA from ST88-14, 90-8, and T265P21 cell lines as a template, exon-specific PCR amplification was applied to the 60 exons of the NF-1 gene in each cell line. The resulting PCR products were analyzed by denaturing high performance liquid chromatography (DHPLC) in order to target abnormal migration patterns heralding possible sequence alterations. For optimal detection of heterozygous mutations, PCR amplification products (amplicons) of the cell lines (putatively containing only the mutated copy of the NF-1 gene) were mixed with amplicons of non-NF-1 cells. Following DHPLC analysis, all abnormally migrating fragments were sequenced using the ABI PRISM® BigDye® and a semiautomatic sequencing kit (PE Biosystems, Foster City, Calif.).

Allelotyping of cell lines using 17q markers-DNA from all cell lines was used as a template for amplification employing four 17 q markers (D17S33, D17S1166, D17S250 and an intronic polymorphic sequence within intron 38). PCR primers and protocols were performed as described in Gutzmer, R., Herbst, R. A., Mommert, S., Kiehl, P., Matiaske, F., Rutten, A., Kapp, A., Weiss, J., Hum Genet 107:357-61 (2000). Analysis of PCR products was carried out on the ABI PRISM310 apparatus, or using an RsaI restriction enzyme digest (for D17S33).

### Cell Culture Procedures

The human NF-1 MPNST cell lines ST88-14, T265P21 and 90-8 and the non-NF-1 human MPNST cell line STS26T were obtained from Dr. Nancy Ratner. T265P21, 90-8, and ST88-14 cells were maintained in RPMI/15% fetal calf serum (FCS) medium and STS26T cells were maintained in DMEM/10% FCS, as described in Weisz, B., et al., supra. Cells were plated at a density of 1×10⁶ cells per 10-cm plate for biochemical and immunoblotting assays, 2.5×10³ cells/well in 24-well plates for cell-growth assays, or on glass cover slips (5×10⁴ cells per 35-mm dish) for labeling of actin cytoskeletal elements. The cells were incubated for 24 hours, treated with the indicated concentration of FTS or with the vehicle (0.1% DMSO) (control) for the times specified in each of the experiments, and then subjected to the various assays as described below. The effect of FTS on cell growth was estimated by direct counting of cells collected from each well, as described in Weisz, B., et al., supra.

### Western Immunoblotting, Ras-GTP and RalA-GTP Assays, and Confocal Microscopy

Unless otherwise indicated, cells were lysed 48 hours after being treated with lysis buffer, as described in Haklai, et al., supra. Lysates containing 50-100 μg protein were subjected to SDS-PAGE followed by Western immunoblotting, as described in Haklai, et al., supra.; Elad-Sfadia, G., Haklai, R., Ballan, E., Gabius, H. J., Kloog, Y., J. Biol. Chem. 277:37169-75 (2002); Paz, A., Haklai, R., Elad, G., Ballab, E., Kloog, Y., Oncogene 20:7486-93 (2000), with one of the following antibodies: 1:2000 pan-Ras Ab-3; 1:10,000 anti-phospho-ERK Ab; 1:2000 anti-ERK Ab; 1:1000 anti-Akt Ab; 1:2000 anti-phospho-Akt Ab; 1:500 antitubulin Ab; 1:5000 anti-RalA Ab; 1:1000 anti-p120 RasGAP Ab; or 1:200 anti-neurofibromin Ab. The immunoblots were then exposed either to 1:7500 peroxidase goat anti-mouse IgG or to 1:2000 peroxidase goat anti-rabbit IgG. Protein bands were visualized by ECL and quantified by densitometry with Image Master VDS-CL (Amersham) using TINA 2.0 software (Ray Tests). To obtain reliable comparisons between replicated experiments for statistical analysis, a standardization procedure was used, in which the density of each given protein band as recorded with its specific Ab, was normalized. Normalization was achieved by collecting and averaging all the data obtained for a given band (for example, for the Ras-GTP band) in a set of experiments, irrespective of cell type or treatment. The standard average density was defined as 1.0, and each of the individually determined bands was related to this value. Accordingly, the normalized value of a single band would be 1.0 if it was equal to the standard value, or would range between values above and below 1.0 (respectively representing values higher and lower than the standard). The normalized values thus obtained were used to calculate means±SD and, to determine, using Student's t-test, the statistical significance of differences between populations. Lysates containing 500 μg protein were used to determine Ras-GTP by the GST-RBD pull-down assay, and this was followed by Western immunoblotting with pan anti-Ras Ab as described in Elad-Sfadia, G., et al., supra. Lysates containing 500 μg protein were used to determine RalA-GTP by the GST-RalBD pulldown assay, followed by Western immunoblotting with anti-RalA Ab [Wolthuis, R. M., Zwartkruis, F., Moen, T. C., Bos, J. L. Ral. Curr. Biol. 8:471-4 (1998)]. Actin cytoskeletal elements were labeled with rhodamine-phalloidin, and digital fluorescence images were collected on a Zeiss LSM 510 confocal microscope fitted with fluorescein and rhodamine filters, as described in Gana-Weisz, M., et al., supra.

### Soft Agar Assays and Animal Experiments

Noble agars (2% and 0.6%; Difco, Sparks, Md.) were prepared in double-distilled water and autoclaved. The 2% agar was melted and mixed with medium (DMEM X2 with 20% FCS, 100 μg/mL penicillin, and 0.1 μg/mL streptomycin) and the mixture (50 μL) was placed in 96-well plates to provide the base agar (at a final concentration of 1%). ST88-14 (5,000 cells/well) and STS26T (15,000 cells/well) were suspended in medium (DMEM X2 mixed with 0.6% agar, and 50 μL of the mixture was plated on the base agar. FTS or vehicle containing DMEM/10% FCS (100 μL) was added to the wells. The plates were incubated for 14-21 days at 37° C. and colonies were then stained with 3-(4,5-dimethylthiazol-2-yl)-2,5-diphenyltetrazolium bromide] (MTT; 1 mg/mL) and photomicrographed. The number of colonies per well was determined using ImagePro software.

ST88-14 cells (5×10⁶) in Matrigel were implanted subcutaneously (s.c.) just above the right femoral joint of nude CD1-Nu male mice (6 weeks old). Two weeks later palpable tumors were observed and the mice were separated randomly into vehicle-treated control and FTS-treated groups. Two types of treatment protocols were used. (1) The first protocol was intraperitoneal (i.p.) administration (0.1 mL) of vehicle, 1 mg/kg, 5 mg/kg or 10 mg/kg FTS (n=6 per group). The FTS solution was prepared in saline containing phosphate buffer as described in Haklai, et al., supra. (2) The second protocol was oral administration of FTS. Mice received vehicle or 20, 40, 60, or 80 mg/kg FTS (n=10 per group). The FTS mixture for oral administration was prepared in PBS/0.5% carboxymethyl cellulose. Mice were sacrificed 6 weeks after the start of the treatment, and tumors were removed and weighed. Student's t-test was used for data analysis.

### Results

### I. NF-1 Associated MPNST Cell Lines Exhibited Pathogenic NF-1 Gene Mutations and Neurofibromin Deficiencies.

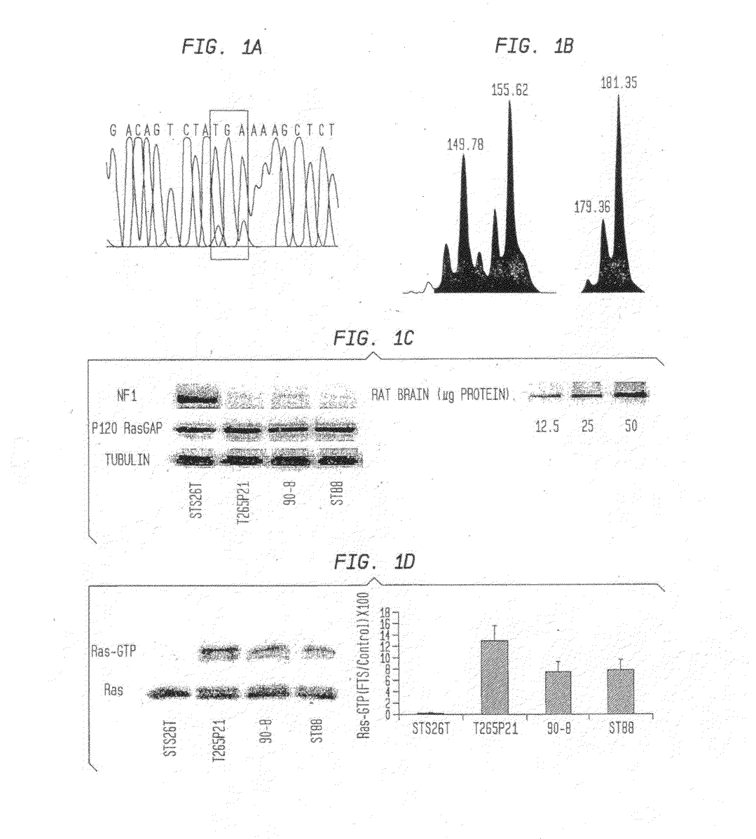

The NF-1-associated cell lines were originally derived from human MPNST cells, and therefore, were assumed to possess an NF-1^{-/-} genotype. In order to define the specific pathogenic inactivating NF-1 mutation in these cell lines, the particular NF-1 gene mutation was confirmed. A heterozygous nonsense mutation (C910T) in codon 304 (R304X) of exon 7 of both 90-8 (FIG. 1A) and ST88-14 (data not shown) cell lines was detected. This pathogenic mutation leading to this exon 7 skipping is well-known. [Wimmer, K., Eckart, M., Stadler, P. F., Rehder, H., Fonatsch, C, Hum Mutat 16:90-1 (2000)]. Another mutation detected in the 90-8 cell line was a seven base-pair deletion in exon 23a of the NF-1 gene (GATCCTT). Thus, these results confirmed that 90-8 cells were deficient in neurofibromin, as demonstrated in earlier reports. [DeClue, J. E., et al., Cell 69:265-13 (1992)]. The ST88-14 cell line, however, contained a pathogenic inactivating mutation, as yet undetected. Additionally, no pathogenic mutation was detected by applying this technique in the T265P21 cell line, and all migration abnormalities detected in this cell line by DHPLC were shown to be silent polymorphisms.

Despite the lack of normal tissue to compare allelic patterns, all NF-1 cell lines consistently showed a pattern of a single dominant allele, and another one that seemed to be a lesser intensity allele, with the three intragenic markers (see FIG. 1B). The D17S250 marker cell line 90-8 showed retention of both alleles (FIG. 1B). The other cell lines showed the same pattern in all four markers—a pattern consistent with allelic loss. These allelotyping results showed that the cell lines originate from monoclonal tumors, but despite their monoclonality, there existed a residual DNA contribution from a non-tumorous tissue, as evidenced by the minimal signal of the “lost allele”.

To establish conclusively that these cell lines were neurofibromin deficient, neurofibromin expression was assessed by Western immunoblot analysis using cell lysates from all four cell lines and a specific anti-neurofibromin Ab. The analysis showed that the non-NF-1-associated STS26T cells expressed significant amounts of neurofibromin, equivalent to the amount of neurofibromin detected in 50 μg of protein from total brain homogenate, which served as a standard (FIG. 1C). In contrast, only small amounts of neurofibromin were detectable in ST88-14, 90-8, and T265P21 cells (FIG. 1C). Immunoblot analysis with anti-p120 RasGAP Ab indicated, nonetheless, that the amounts of p120 RasGAP in ST88-14, 90-8, and T265P21 cells were indistinguishable from the amounts in STS26T cells (FIG. 1C).

II. Steady-State Levels of Ras-GTP Correlated Positively with Neurofibromin Deficiency and Growth Inhibition by FTS.

To determine whether the steady-state levels of active Ras in the MPNST cell lines correlated with neurofibromin deficiency, the GST-Ras binding domain of Raf-1 (RBD) pull-down assay and Western immunoblotting with anti-Ras Ab was performed. The amounts of total Ras in all cell lines were clearly comparable, but the amounts of Ras-GTP varied (FIG. 1D). Ras-GTP (means±SD, n=6) in the non-NF-1 STS26T cells was very low [0.17±0.14% of the total Ras protein), but was far higher in the TS265P21, 90-8, and ST88-14 cells [3±2.5%, 7.5±1.8%, and 7.7±1.8% of the total Ras protein, respectively (FIG. 1D)]. Thus, the relatively large amounts of Ras-GTP detected in the MPNST cell lines and the relatively small amounts in the non-NF-1 MPNST (FIG. 1D) correlated well with the observed neurofibromin deficiency (FIG. 1C).

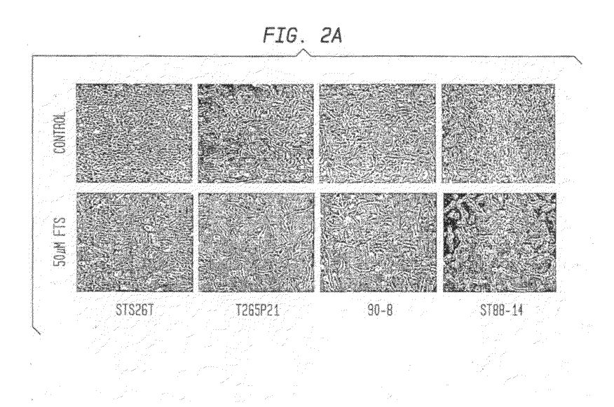

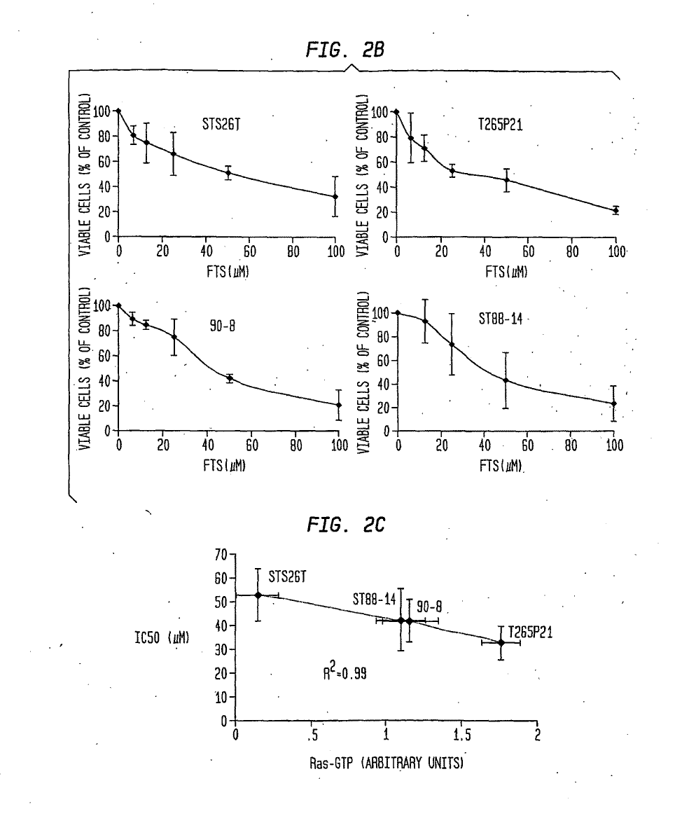

To determine the effects of the Ras inhibitor FTS on the growth rates of the NF-1 cell lines ST88-14, 90-8, T265P21 and the non-NF-1 STS26T cells, the cells were grown for two days in the absence and in the presence of 50 μM FTS, and then photomicrographed. Typical photomicrographs of control and FTS-treated cells (FIG. 2A) demonstrated a drug-induced reduction in cell number. Under these conditions there was no significant cell death. Additionally, cells were grown for three days and then detached, collected, and counted as described in Methods to determine dose-dependent inhibition by FTS. Treatment of the cells with different concentrations of FTS (12.5-100 μM) induced a dose-dependent decrease in cell number, with estimated IC₅₀ values of 35±7, 42±13, 42±9, and 53±10 μM (means±SD, n=4) in T265P21, ST88-14, 90-8, and STS26T cells, respectively (FIG. 2B). Sensitivity to FTS as determined by these values showed good inverse correlation with apparent amounts of Ras-GTP in the studied cell lines: the larger the amount of Ras-GTP, the lower the IC₅₀ value (FIG. 2C).

### III. FTS Down-Regulated Ras-GTP and Inhibited Ras Signaling in NF-1 Cells.

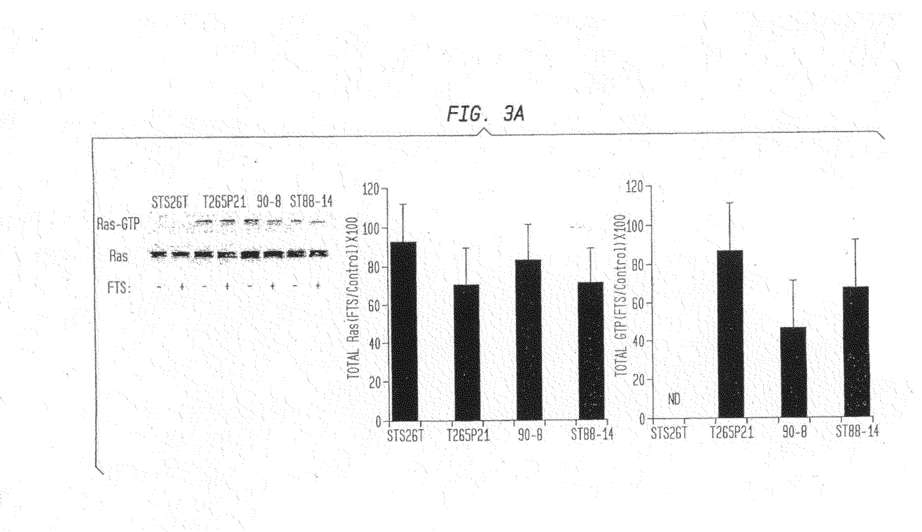

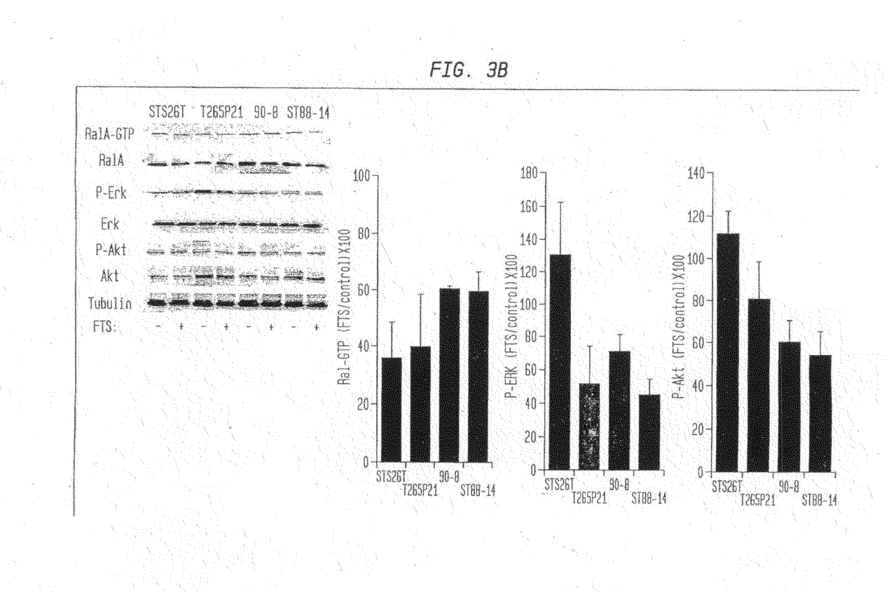

The effects of FTS on steady-state levels of Ras and on Ras-GTP levels in each of the studied cell lines were examined to detect whether inhibition of the cellular growth of MPNST NF-1 cells by FTS might be attributable to FTS-induced inhibition of active Ras and its downstream signals. Accordingly, all four cell lines were treated with the vehicle (control) or with 75 μM FTS for 48 hours. FTS treatment induced relatively small (15-30%) but significant (P<0.05) reductions in Ras levels in all cell lines (FIG. 3A). Even greater reductions in the steady-state levels of Ras-GTP (25%-55%, P<0.03) were observed in TS265P21, 90-8, and ST88-14 cells (FIG. 3A). The low basal levels of Ras-GTP in STS26T cells precluded any attempt at accurate analysis of Ras-GTP in the presence of FTS, although total Ras was clearly decreased in these cells, as it was in the NF-1 cell lines (FIG. 3A). These experiments showed that FTS inhibited Ras which down-regulated active Ras in NF-1 cells. FTS (75 μM, 48 hours) had no effect on the amounts of p120 RasGAP in the NF-1 and non-NF-1 cells (not shown), indicating that the decrease in active GTP-bound Ras resulted from a direct effect of the Ras inhibitor on active Ras and was not achieved indirectly by increasing p120 RasGAP.

Next, to determine whether down-regulation of active Ras by FTS was accompanied by inhibition of Ras signaling, the effect of FTS on steady-state levels of phospho-ERK, phospho-Akt, and RalA-GTP as read-outs of the three prominent Ras pathways, Ras/Raf/MEK/ERK, Ras/PI3K/Akt, and Ras/RalGEF/RalA, respectively, was examined. [Marshall, C. J., Curr. Opin. Cell Biol. 8:197-204 (1996); Bar-Sagi, D., Hall, A., Cell 103:227-238 (2000)]. The results of these experiments showed that FTS caused a significant reduction in phospho-ERK, phospho-Akt, and RalA-GTP in all NF-1 cell lines (FIG. 3B), but did not affect the amounts of total ERK, total Akt, or total RalA. FTS also caused a significant reduction in RalA-GTP in the non-NF-1 STS26T cells (FIG. 3B) and a small (10%-20%), unexplained increase in phospho-ERK and phosphor-Akt. Thus, down-regulation of active Ras in NF-1 cells was accompanied by a reduction in activation of three prominent Ras downstream pathways.

### IV. FTS Restored an Attenuated Ras Signal-Termination in NF-1 Cells.

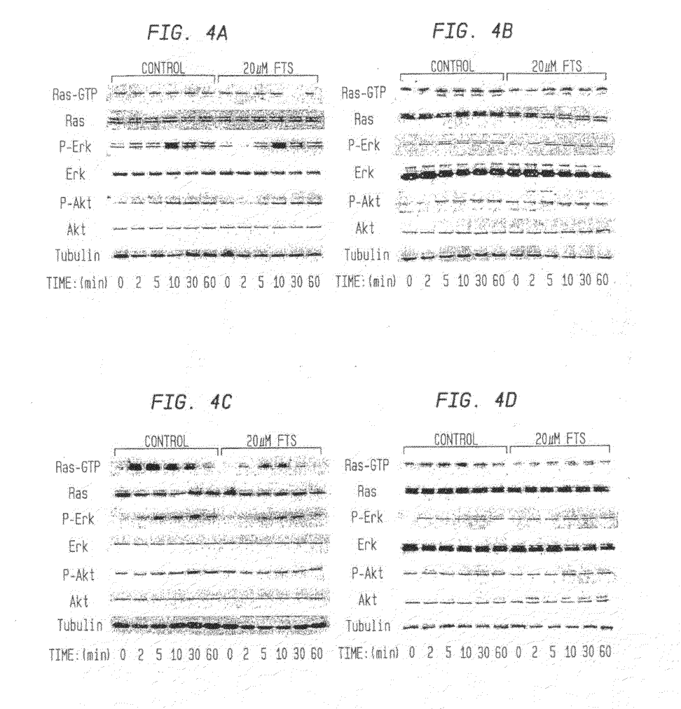

To determine whether the Ras signaling in NF-1 cells would be relatively prolonged owing to the deficiency of neurofibromin, and whether the Ras inhibitor FTS would shorten that signal, a detailed kinetic study was performed. First, the time courses of serum-stimulated GTP loading of Ras and serum-stimulated activation of signals downstream of Ras were determined. The cells were treated with FTS and serum-starved (0.5% serum) for 24 hours before being stimulated with 10% serum for the indicated time periods. Results of a typical experiment with the non-NF-1-derived cells, STS26T, are shown in FIG. 4A. As depicted, stimulation with 10% serum induced a strong and rapid increase (within 2 minutes) in Ras-GTP, lasting for at least 10 minutes. The increase was transient, however, and by 30 or 60 minutes after serum stimulation the amounts of Ras-GTP were substantially reduced (FIG. 4A). This serum-stimulated increase in Ras-GTP was strongly inhibited, by 20 μM FTS (FIG. 4A). Notably, unlike in STS26T cells grown in the presence of serum (steady-state conditions), where Ras-GTP was undetectable (FIG. 1D), a robust increase in Ras-GTP in the serum-starved cells upon stimulation with serum was observed.

Phospho-ERK and phospho-Akt in STS26T cells also showed a serum-stimulated transient increase, which was slightly delayed (by 3-5 minutes and 5-10 minutes, respectively) compared to that observed in Ras-GTP. This delay may be accounted for by the fact that phospho-ERK and phospho-Akt are downstream targets of Ras [Marshall, C. J., Curr. Opin. Cell. Biol. 8:197-204 (1996); Bar-Sagi, D., Hall, A., 103:227-238 (2000)]. Nonetheless, as in the case of serum activation of Ras, the increases in phospho-ERK and phospho-Akt levels were transient, and after 60 minutes were substantially reduced (FIG. 4A). FTS also inhibited the serum-stimulated increase in phospho-ERK and in phospho-Akt (FIG. 4A). Under these conditions FTS had no effect on the total amounts of Ras, ERK, or Akt. Thus, serum stimulation of STS26T cells resulted in transient activation of Ras and its downstream signals, and this was inhibited by FTS.

Next, we conducted a similar set of experiments using NF-1 T265P21 cells (FIG. 4B), ST88-14 cells (FIG. 4C), and 90-8 cells (FIG. 4D). In each of these cell lines, we observed a strong and rapid (within 2 minutes) serum-stimulated increase in Ras-GTP, similar to that observed in the non-NF-1 cells. However, in marked contrast to the observation in the latter cells, the serum-stimulated increase in Ras-GTP levels in the NF-1 cells was maintained for at least 30 minutes, by which time Ras-GTP levels in the non-NF-1 cells had already declined. Similarly, the signals to ERK and Akt in the NF-1 cells were more prolonged (FIG. 4B-D) than in the non-NF-1 cells (FIG. 4A), and phospho-ERK and phospho-Akt levels were still relatively high even 60 minutes after serum stimulation. Notably, in all the NF-1 cell lines FTS not only reduced the serum-stimulated increases in Ras-GTP, phosphor-ERK, and phospho-Akt levels, but also shortened the duration of the signals (FIG. 4B-D). Thus, in the presence of FTS the duration of the Ras signal in the NF-1 cells (5-10 minutes) was similar to that observed in the non-NF-1 cells.

### V. FTS Affected Cytoskeleton-Reorganization in NF-1 Cells.

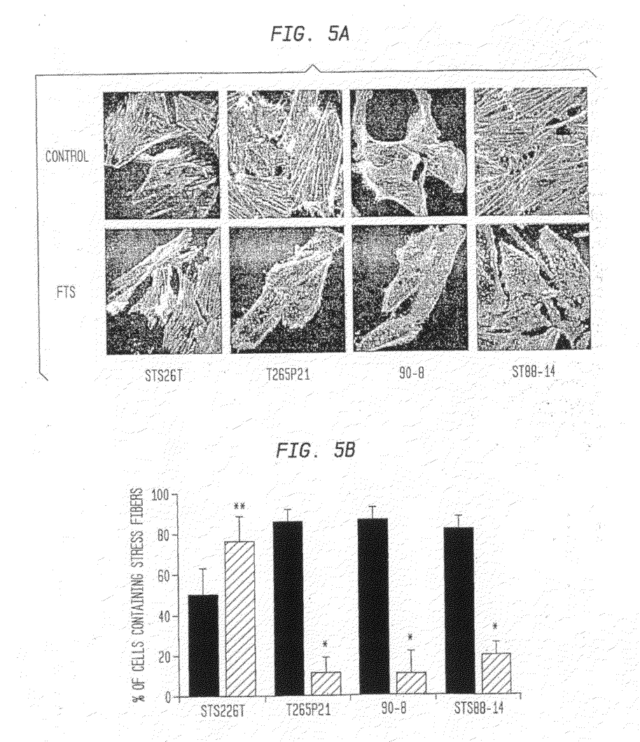

Earlier reports demonstrated that loss of neurofibromin induced excessive formation of actin stress, fibers in HT1080 and HeLa cells. [Ozawa, T., et al. J. Biol. Chem. 280:39524-33 (2005)]. Thus, to detect whether stress fiber formation caused by neurofibromin deficiency was observed in the MPNST NF-1 cells and whether this phenotype could be reversed by FTS, a comparative analysis of cytoskeleton reorganization in MPNST cells and in non-NF-1 STS26T MPNST cells was performed. Both control and FTS-treated cells (75 μM) were stained with rhodamine-labeled phalloidin, which binds to polymeric F-actin. The cells were examined by confocal fluorescence microscopy and the images collected as described in Rotblat, B., Niv, H., Andre, S., Kaltner, H., Grabius H. J., Kloog, Y., Cancer Res. 64:3112-8 (2004). Typical images obtained from experimentation are shown in FIG. 5A. The most prominent actin structures found in the untreated-controls of all cell lines were stress fibers (FIG. 5A). However, a small but significant difference between the non-NF-1 and the NF-1 cells in the numbers of cells expressing stress fibers was observed. Relative to the total cell number, the proportion of cells with stress fibers was 86%±6.7%, 82%±6.1%, and 87%±6.4% in T265P21, ST88-14, and 90-8 cells, respectively, and significantly lower in the non-NF-1 STS26T cells (50±11.8%; P<0.05; FIG. 5B). Consistent with earlier observations in non-NF-1 Ras-trans formed cells, FTS induced a significant increase of 27%±11.9% (mean±SD, n=3, P<0.05) in stress-fiber formation in the non-NF-1 STS26T cells (FIG. 5). In marked contrast, FTS induced a significant decrease in the numbers of cells with stress fibers (from 82%-87% to 11%-20%) in the NF-1 cell lines T265P21, ST88-14, and 90-8 (FIG. 5B). Thus, nearly all of the NF-1 cells lost their stress fibers. These results demonstrated reversal of the transformed phenotype of the NF-1 cells by the Ras inhibitor FTS.

### VI. FTS Inhibited the Anchorage-Independent Growth of ST88-14 and STS26T NF-1 Cells.

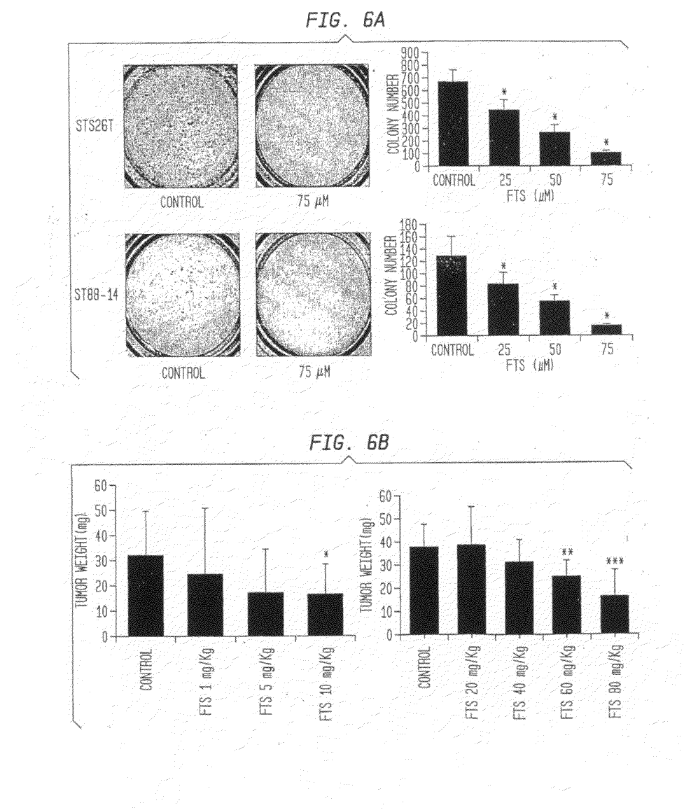

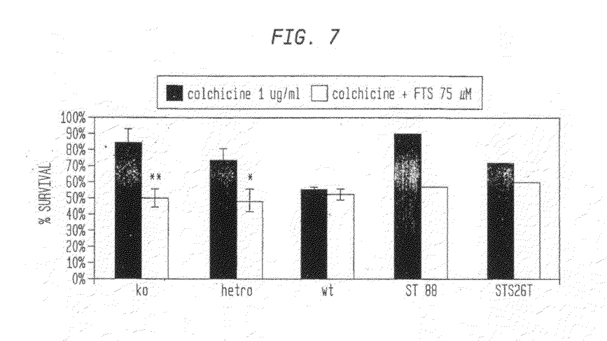

To determine whether FTS inhibited the transforming activity associated with NF-1-deficiency and to determine the effect of FTS on anchorage-independent growth, soft agar assays were performed. ST88-14 and STS26T cells were plated in soft agar and then treated with 0.1% DMSO as a control or with FTS (25 μM, 50 μM, or 75 μM) and grown for 3 weeks or 2 weeks, respectively. Results of a typical experiment (FIG. 6A) demonstrated that control ST88-14 and STS2-6T cells developed approximately 130 and 670 colonies per plate, respectively, within this time period. FTS inhibited colony formation of both cell lines; in the presence of 50 μM FTS, for example, colony formation was inhibited by 60% in STS26T cells and by 57% in ST88-14 cells. In the presence of 75 μM FTS, colony formation was inhibited by 84% and 87% in ST88-14 and STS26T cells, respectively. Unlike ST88-14 and STS26T, the other two MPNST cell lines, T265P21 and 90-8, did not form colonies in soft agar.

### VII. FTS Inhibited Timor Growth in ST88-14 Cells in Nude Mice.

Earlier reports demonstrated that nude mice implanted with ST88-14 cells developed tumors. [Ozerdem, U., Angiogenesis 7:307-11 (2004)]. Thus, to determine whether FTS inhibited tumor growth in the nude mouse model, two types of treatments were preformed. The first was i.p. administration of 1, 5, or 10 mg/kg FTS, as earlier described. [Weisz, B., et al., supra.]. The second was oral administration, which required higher FTS doses of 20, 40, 60, and 80 mg/kg. Six weeks after the treatment the mice were sacrificed and tumors were removed and weighed. As illustrated by FIG. 6B, both the i.p. and the oral FTS treatments caused dose-dependent inhibition of tumor growth: the i.p. protocol yielded a significant inhibition of 49% (P<0.05) at a dosage of 10 mg/kg FTS, while the oral protocol yielded a significant inhibition of 58% (P<0.01) at a dosage of 80 mg/kg.

### Discussion

These experiments strongly supported the notion that active Ras, in the context of neurofibromin deficiency, played an important role in the etiology and clinical manifestations of NF-1. First, the MPNST cells (ST88-14, T265P21, and 90-8) chosen for experimentation were confirmed to be genetically biallelic, NF-1 inactive, and deficient in neurofibromin protein. Next, the expression of large amounts of Ras-GTP in these cells was confirmed, and the large amount of Ras-GTP was associated with neurofibromin deficiency. The results further indicated that FTS reduced the steady-state levels of Ras-GTP in the NF-1 cells, which resulted in the inhibition of three major down-stream targets of Ras: ERK, Akt, and RalA. Finally, in vivo, FTS inhibited the anchorage-dependent growth of the NF-1 cells, attenuated their anchorage-independent, growth in soft agar, and inhibited NF-1 tumor growth in a nude mouse model.

Sensitivity, of the NF-1 cells to FTS, as revealed by growth-inhibition curves, showed good inverse correlation with the apparent amounts of Ras-GTP in the cells: the greater the amount of Ras-GTP, the lower the IC₅₀ value. Our detailed kinetic analysis of serum-stimulated non-NF-1 MPNST (STS26T) and NF-1 cells disclosed that Ras signals in NF-1 cells took longer to fade leading to prolonged activation of Ras, as well as of ERK, Akt, and RalA. Thus, neurofibromin deficiency appeared to prolong Ras signaling. Our results strongly suggested that the relatively high steady-state levels of active Ras exhibited by the NF-1 cells were attributable to the lack of neurofibromin GTPase activity. Thus, once these neurofibromin-deficient cells received growth-factor signals, the receptor-mediated GTP loading was terminated at a relatively slow pace, weakening the signal termination. Signal termination in the non-NF-1 STS26T cells proceeded at a higher rate likely because of the mechanisms that allow rapid recovery of NF-1 [Cichowski, K., et al., Genes Dev. 17:449-54 (2003)].

Furthermore, FTS not only reduced the serum-stimulated increases in Ras-GTP, phospho-ERK, and phospho-Akt in all NF-1 cell lines, but also forced a shorter signal. Thus, in the presence of FTS the duration of the Ras signal in the NF-1 cells (5-10 minutes) was similar to that observed in the non-NF-1 cells. Therefore, with respect to Ras activation, neurofibromin deficiency can apparently be corrected with FTS.

In addition, the results also revealed that FTS induced the complete disappearance of actin stress fibers in NF-1 cells. Ostensibly, this phenomenon was associated with the increased amounts of Ras-GTP in the NF-1 cells since nearly all NF-1 cells possessed strong actin stress fibers, in sharp contrast to the non-NF-1 cell line.

Overall, FTS induced reversal of the aberrant Ras-associated transformed phenotype in NF-1 cells and also attenuated NF-1 tumor growth in FTS-treated animals.

### Example 2

### Experimental Design

The purpose of these in vitro experiments was to examine the survival of NF-1^{-/-}, NF-1^{+/-}, NF-1^{+/+} SV 40 MEF cells, ST-88 cells, non-NF-1 human MPNST STS26T cells after exposure to colchicine in the presence of FTS.

Results demonstrated that colchicine in the presence of FTS enhanced colchicine-induced cell death by decreasing cell survival in NF-1^{-/-} and NF-1^{+/-} SV 40 mouse embryonic fibroblast (MEF) cells and also in neurofibromin-deficient human ST-88 cells. In contrast, colchicine-induced cell death was not enhanced in NF-1^{+/+} SV 40 MEF cells or in the non-NF-1 human MPNST cell line STS26T, which express neurofibromin.

### Materials and Methods

FTS was provided by Concordia Pharmaceuticals, Inc. (Ft. Lauderdale, Fla.). Colchicine was obtained from Biological Industries (Kibbutz Beit Haemek 2115, Israel (cat #12-003-1C). All reagents were purchased from Sigma (St. Louis, Mo.) unless otherwise indicated.

### Cell Culture

Human NF-1 ST88-14 MPNST, non-NF-1 STS26T MPNST, and the different primary and SV40-immortalized NF-1 genotypes (NF-1^{-/-}, NF-1^{+/-}, and NF-1^{+/+}) of mouse embryonic fibroblasts (MEFs) were grown in high-glucose Dulbecco's modified Eagle's medium (DMEM) supplemented with 10% heat-inactivated fetal calf serum (both from Biological Industries, Beit Ha Emek, Israel) with 0.0004% β-mercaptoethanol added to the MEF growth medium only.

### Transgenic NF-1 Mice and Preparation of Primary Mouse Embryonic Fibroblasts

C57BL/6J mice with a targeted NF-1 gene allele were obtained from Dr. Nancy Ratner (University of Cincinnati Medical School, Cincinnati, Ohio). [Brannan, C.I., Perkins, A. S., Vogel, K. S., Ratner, N., Nordlund, M. L., et al., Genes Dev 8:1019-1029 (1994)]. To obtain the three NF-1 genotypes (NF-1^{-/-}, NF-1^{+/-}, and NF-1^{+/+}) NF-1^{+/-} mice were mated and checked for the presence of a copulatory plug. To obtain embryos, pregnant mice were killed after 11.5 days (the day the plug was observed was designated as day 0.5). Embryos were transferred to a sterile dish, washed in PBS, their internal organs were removed, and the rest of the embryo was chopped into small pieces that were then incubated with trypsin/EDTA for 40 min at 37° C. and 8% CO₂, followed by centrifugation at 1,000×g. The resulting pellets were resuspended in growth medium and the cells were seeded in 10-cm plates. After 4 h the medium was changed and the attached surviving fibroblasts were allowed to grow further. The genotypes of the different embryos were determined by PCR using the appropriate primer set as described in Brannan, C. I., et al., supra.

### Generation of the SV40-Immortalized MEFs

The three primary NF-1 MEF genotypes were immortalized by transfection with SV40 whole genome DNA (a gift from Dr. Atan Gross, The Weizmann Institute of Science) using the lipofectamine transfection reagent (InvitroGen Life Technologies, Paisley, UK). One day before transfection, primary MEFs were seeded at a density of 2×10⁵ cells per well in 6-well plates. To each dish we added 1 ml of DNA-lipofectamine mixture, 0.1 μg SV40 whole genome DNA, and 10 μg of lipofectamine in 1 ml of OptiMEM (InvitroGen), according to the manufacturer's instructions. The cells were split 24 h after transfection and seeded in 10-cm² dishes at a density of 1×10³ to 1×10⁴ cells per dish. Immortalized clones from each NF-1 genotype were collected 14 days after transfection. For each of the three genotypes at least three different clones, obtained from different batches of primary cultures, were analyzed for their Ras signaling pathways and susceptibility to apoptosis, and all yielded similar results. For further analysis, clones c1 (NF-1^{-/-}), c7 (NF-1^{+/-}) and c7 (NF-1^{+/+}) were chosen as representative clones for the immortalized NF-1 MEF genotypes and were designated SV40 NF-1^{-/-} MEF, SV40 NF-1^{+/-} MEF, and SV40 NF-1^{+/+} MEF, respectively.

### Assessment of Cell Survival Using MTT

The numbers of live cells in the 96-well plates (5×10³ cells per well) were determined by the 3-(4,5-dimethylthiazol-2-yl)-2,5-diphenyltetrazolium bromide (MTT) assay, as described in Lindenboim, L., Haviv, R., Stein, R., J Neurochem 64:1054-1063 (1995). Briefly, MTT was dissolved in PBS at a concentration of 5 mg/ml. From this stock solution, 10 μl per 100 μl of medium was added to each well, after which the plates were incubated at 37° C. for 4 h. Acid-isopropanol (100 μl of 0.04 M HCl in isopropanol) was then added to the well and mixed in. After 15 minutes at room temperature, the plates were read on a microELISA reader at a test wavelength of 540 nm and a reference wavelength of 690 nm.

### Results

### FTS Promotes Colchicine-Induced Death in Neurofibromin Deficient Cells

In order to examine the role of colchicine in combination with FTS on neurofibromin levels, SV40 immortalized MEF cells NF-1^{-/-} (ko), NF-1^{+/-} (hetero), and NF-1^{+/+} (wt) SV40 MEFs, and human ST-88 (neurofibromin deficient) and non-NF-1 human MPNST STS26T (normally expressing neurofibromin) cells were used. C57BL/6J mice with a targeted NF-1 gene allele (NF-1^{+/-}) were mated to obtain primary MEF cultures of the three NF-1 genotypes. These cultures were then utilized to establish SV40-immortalized MEFs (“SV40 MEFs”) of the three NF-1 genotypes. Neurofibromin levels confirmed that NF-1^{-/-} cells had no neurofibromin, NF-1^{+/-} cells had relatively small amounts of neurofibromin, and NF-1^{+/+} cells had relatively large amounts of neurofibromin (data not shown).

NF-1^{-/-} (ko), NF-1^{+/-} (hetero), and NF-1^{+/+} (wt) SV40 MEFs, and human ST-88 and STS26T were treated with 75 μM FTS or its vehicle (0.1% DMSO) for 24 h and were then left untreated or were treated with colchicine for 4.5 h. Cell viability was determined by the MTT assay.

The results in FIG. 5 showed that colchicine alone had only a small effect on cell survival in NF-1^{-/-} and NF-1^{+/-} (neurofibromin deficient) cells, while the combination of FTS with colchicine decreased cell survival, i.e., FTS enhanced the colchicine-induced cell death. Combined treatment with FTS also enhanced colchicine-induced cell death in neurofibromin-deficient human ST-88 cells. In contrast, in NF-1^{+/+} cells, colchicine alone induced death while combined treatment with FTS did not have an enhanced effect on colchicine-induced cell death. Similarly, in STS26T cells, which express neurofibromin, cell death was not enhanced by administering colchicine in the presence of FTS.

### Representative Formulations of Oral Dosage Forms

### Example 3

### Tablets of FTS (200 mg) and Colchicine (0.50 mg)

FTS active pharmaceutical ingredient (2000 g), colchicine active pharmaceutical ingredient (5.0 g), microcrystalline cellulose (2000 g), croscarmellose sodium (200 g), and magnesium stearate (100 g) are blended to uniformity and compressed into tablets weighing 430.50 mg. Assuming a 5% loss on material transfers and tablet press start-up, adjustment, and shut down, approximately 9,500 tablets of FTS 200 mg/colchicine 0.50 mg are yielded.

By adjusting fill weight, tablet size, excipient amounts, or the relative amounts of the two actives, other tablet strengths are prepared.

### Example 4

### Tablets of FTS (200 mg) and Colchicine (0.25 mg)

FTS active pharmaceutical ingredient (2000 g), colchicine active pharmaceutical ingredient (2.50 g), microcrystalline cellulose (1500 g), starch (500 g), and magnesium stearate (25 g) are blended to uniformity and compressed into tablets weighing 402.8 mg. Assuming a 5% loss on material transfers and tablet press start-up, adjustment, and shut down, approximately 9,500 tablets of FTS 200 mg/colchicine 0.25 mg are yielded.

By adjusting fill weight, tablet size, excipient amounts, or the relative amounts of the two actives, other tablet strengths are prepared.

### Example 5

### Capsules of FTS (100 mg) and Colchicine (0.25 mg)

FTS active pharmaceutical ingredient (2000 g), colchicine active pharmaceutical ingredient (5.0 g), lactose (2000 g), microcrystalline cellulose (1000 g), and amorphous colloidal silicon dioxide (15 g) are blended to uniformity and filled into hard shell gelatin capsules. Assuming a 5% loss on material transfers and encapsulating machine start-up, adjustment, and shut down, approximately 19,000 capsules of FTS 100 mg/colchicine 0.25 mg are yielded.

By adjusting fill weight, capsule size, excipient amounts, or the relative amounts of the two actives, other capsule strengths are prepared.

The publications cited in the specification, patent publications and non-patent publications, are indicative of the level of skill of those skilled in the art to which this invention pertains. All of these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1-22. (canceled)

23. A method of treating a human having neurofibromatosis, comprising administering to the human an effective amount of farnesylthiosalicylic acid (FTS) or an analog thereof as represented by the formula: 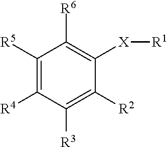
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof.

24. The method of claim 23, wherein the human is administered FTS.

25. The method of claim 23, wherein the human is administered S-geranyl,geranyl-thiosalicylic acid (GGTS).

26. The method of claim 23, wherein FTS or its analog is administered orally.

27. A method of treating a human having neurofibromatosis, comprising administering to the human an effective amount of farnesyl-thiosalicylic acid (FTS) or an analog thereof as represented by the formula: 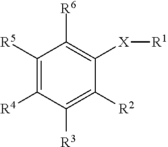
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof, and
colchicine.

28. The method of claim 27, wherein the human is administered FTS.

29. The method of claim 27, wherein the human is administered GGTS.

30. The method of claim 27, wherein FTS or its analog and the colchicine are contained in separate dosage forms.

31. The method of claim 27, wherein FTS or its analog and the colchicine are administered orally.

32. A composition useful in the treatment of neurofibromatosis, comprising effective amounts of a compound which is FTS or an analog thereof as represented by the formula: 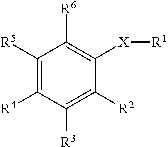
wherein
R¹ represents farnesyl, geranyl or geranyl-geranyl;
R² is COOR⁷, or CONR⁷R⁸, wherein R⁷ and R⁸ are each independently hydrogen, alkyl or alkenyl;
R³, R⁴, R⁵ and R⁶ are each independently hydrogen, alkyl, alkenyl, alkoxy, halo, trifluoromethyl, trifluoromethoxy, or alkylmercapto; and
X represents S; or a pharmaceutically acceptable salt thereof, and
colchicine; and
a pharmaceutically acceptable carrier.

33. The composition of claim 32, wherein the composition comprises FTS.

34. The composition of claim 32, which is in the form of a tablet.

35. The composition of claim 32, which is in the form of a capsule.


SEQUENCE LISTING (1 sequence)

SEQ ID NO: 1 - DNA, 20 bp, Artificial Sequence
  Description of Artificial Sequence Synthetic oligonucleotide
gacagtctatgaaaagctct